(19) United States
(12) Patent Application Publication
(10) Pub. No.: US 2003/0114840 A1
(43) Pub. Date: Jun. 19, 2003

(54) Eye positioning system and method

(76) Inventor: Haizhang Li, Orlando, FL (US)

Correspondence Address: Jacqueline E. Hartt, Allen, Dyer, Doppelt, Milbrath & Gilchrist, P.A., 255 South Orange Avenue, Suite 1401, P.O. Box 3791, Orlando, FL, 32802-3791, US

(21) Appl. No.: 10/020,589

(22) Filed: Dec. 14, 2001

(51) Int. Cl. A61B 18/18

(52) U.S. Cl. 606/5

(57) ABSTRACT

A system for positioning an eye for laser surgery includes a first and a second line generator positioned, respectively, to emit a first line and a second line orthogonal to the first line, the line generators positioned in a predetermined relation to a laser surgical system. A position of the eye is adjustable along an axis that is substantially perpendicular to the lines to achieve a positioning wherein the lines form a cross. The location of the cross is a preferred position for the eye relative to the laser surgical system for the laser surgery. The method of the present invention includes generating the orthogonal lines of radiation and adjusting a position of an eye to be along an axis substantially perpendicular to the lines to form a cross on a cornea of the eye in a plane.

### FIELD OF INVENTION

The present invention generally relates to objective measurements and surgical correction of a human eye and, in particular, to systems and methods for ensuring a correct positioning of the eye for surgery.

### BACKGROUND

Laser surgery on the eye using laser in situ keratomileus (LASIK) is a common type of laser vision correction procedure. It has proven to be an extremely effective outpatient procedure for a wide range of vision corrective prescriptions. The use of an excimer laser allows for a high degree of precision and predictability in shaping the cornea of the eye. Prior to the LASIK procedure, measurements of the eye are made to determine the amount of corneal material to be removed from various locations on the corneal surface so that the excimer laser can be calibrated and guided for providing the corrective prescription previously determined by measurement.

Procedures such as LASIK require precise alignment between the eye and the corrective laser beam. At present the patient is requested to focus on a fixation target such as a light-emitting diode (LED), but holding the eye steady during surgery may prove difficult.

Thus there is a need to provide an accurate reference for orienting the eye for surgery.

### SUMMARY OF INVENTION

It is therefore an object of the present invention to provide a system and method for positioning an eye for surgery.

It is a further object to provide such a system and method that are substantially noninvasive.

It is another object to provide such a system and method that provide a continuous indication of alignment.

These and other objects are achieved by the present invention, a first aspect of which includes a system for positioning an eye for laser surgery. The system comprises a first and a second line generator positioned, respectively, to emit a first line and a second line orthogonal to the first line. The first and the second line generators are further positioned in a predetermined relation to a laser surgical system.

Means for adjusting a position of the eye are positioned along an axis that is substantially perpendicular to the first and the second line. This location is for achieving a positioning wherein the first and the second line form a cross. The location of the cross comprises a preferred position for the eye relative to the laser surgical system for the laser surgery.

The method of the present invention comprises the steps of generating a first and a second line of radiation, wherein the first line is substantially orthogonal to the second line. The first and the second lines are directed to a position having a predetermined relation to a laser surgical system. A position of an eye is then adjusted along an axis substantially perpendicular to the first and the second line to achieve a positioning wherein the first and the second line form a cross on a cornea of the eye in a plane. This plane comprises a preferred position of the eye relative to the laser surgical system for laser surgery.

In an alternate embodiment, the patient bed can remain stationary, and the surgical system translated to the desired position as determined by the system of the present invention.

The features that characterize the invention, both as to organization and method of operation, together with further objects and advantages thereof, will be better understood from the following description used in conjunction with the accompanying drawing. It is to be expressly understood that the drawing is for the purpose of illustration and description and is not intended as a definition of the limits of the invention. These and other objects attained, and advantages offered, by the present invention will become more fully apparent as the description that now follows is read in conjunction with the accompanying drawing.

### BRIEF DESCRIPTION OF THE DRAWING

FIG. 1 is a schematic illustration of the optical system of the present invention.

FIG. 2 is a schematic illustration of the surgical system incorporating the optical system of FIG. 1.

### DETAILED DESCRIPTION OF THE PREFERRED EMBODIMENT

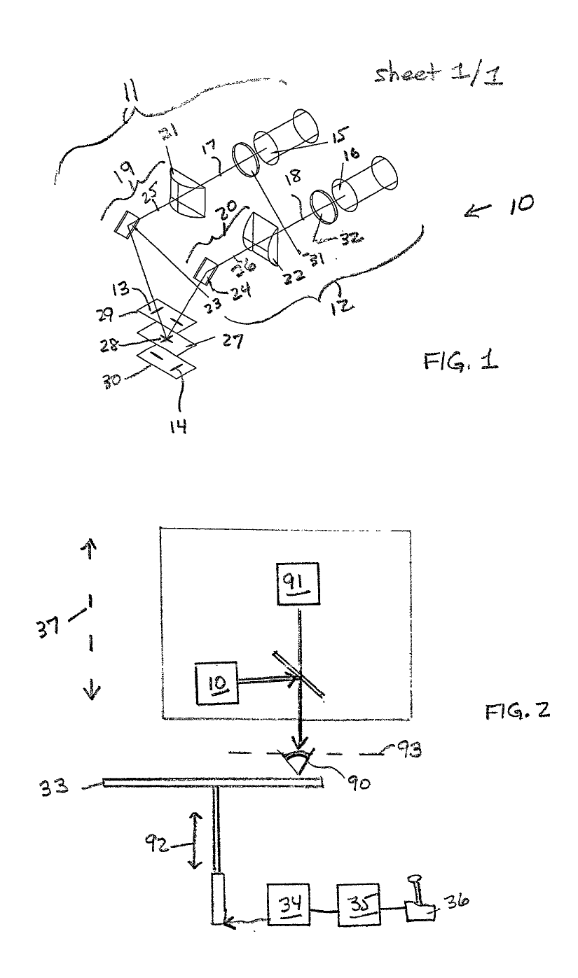

A description of the preferred embodiments of the present invention will now be presented with reference to FIGS. 1 and 2.

The system 10 of the present invention is for positioning an eye 90 for laser surgery, such as, but not intended to be limited to, LASIK surgery. The system 10 comprises (FIG. 1) a first 11 and a second 12 line generator that are positioned, respectively, to emit a first line 13 and a second line 14 that is orthogonal to the first line 13. The first 11 and the second 12 line generator are further positioned in a predetermined relation to a laser surgical system 91.

In a preferred embodiment the line generators 11,12 comprise a first 15 and a second 16 source, for example, laser modules, for forming a first 17 and a second 18 beam of radiation. A first 19 and second 20 optical train transforms the first 17 and the second 18 beam into the first 13 and the second 14 line. The first 19 and the second 20 optical trains comprise in a preferred embodiment a first 21 and a second 22 cylindrical lens downstream of the first 15 and the second 16 laser source. The optical trains 19,20 further comprise a first 23 and a second 24 mirror downstream of the first 21 and the second 22 cylindrical lens, respectively. The first 23 and the second 24 mirror are oriented for forming the lines 13,14 from the beams 25,26 emerging from the first 21 and the second 22 cylindrical lens. A preferred position 27 is indicated wherein a cross 28 is formed between the lines 13,14; positions above 29 and below 30 the preferred position are shown, wherein the lines 13,14 are disjoint.

The optical trains 19,20 additionally comprise a first 31 and a second 32 filter positioned between the first 15 and the second 16 laser source and the first 21 and the second 22 cylindrical lens.

Another aspect of the system (FIG. 2) comprises means for adjusting a relative position of the eye 90 along an axis 92 that is substantially perpendicular to the first 13 and the second 14 line. The adjusting means are for achieving the positioning wherein the first 13 and the second 14 line form the cross 28, which comprises a preferred position for the eye 90 relative to the laser surgical system 91 for laser surgery. In particular, the cross 28 is desired to be formed at a corneal eye plane 93 at which surgery is to be performed. If the lines 13,14 do not form a cross 28, then the eye position is not correct, which is indicated by the lines' 13,14 being disjunct in FIG. 1 at positions 29,30.

In a preferred embodiment of the present invention, the adjusting means comprises a patient bed 33 that is in mechanical contact with a means for moving the bed 33 along a substantially vertical axis 92. The moving means comprises, for example, a hydraulic lift 34, a motor 35 in activating contact with the lift 34, and a controller 36, such as a joystick or other implement known in the art, for controlling the motor 35.

Alternatively, the patient bed 33 can remain stationary, and the surgical system 91 translated as shown by the dotted double-headed arrow 37 to the desired position 27 as determined by the system 10 of the present invention.

In the foregoing description, certain terms have been used for brevity, clarity, and understanding, but no unnecessary limitations are to be implied therefrom beyond the requirements of the prior art, because such words are used for description purposes herein and are intended to be broadly construed. Moreover, the embodiments of the apparatus illustrated and described herein are by way of example, and the scope of the invention is not limited to the exact details of construction.

Having now described the invention, the construction, the operation and use of preferred embodiments thereof, and the advantageous new and useful results obtained thereby, the new and useful constructions, and reasonable mechanical equivalents thereof obvious to those skilled in the art, are set forth in the appended claims.

What is claimed is:

1. A system for relatively positioning an eye and a surgical system for laser surgery comprising:
a first and a second line generator positioned, respectively, to emit a first line and a second line orthogonal to the first line, and further positioned in a predetermined relation to a laser surgical system; and
means for adjusting a relative position of the first and the second line generators relative to the eye to achieve a preferred position for laser surgery.

2. The system recited in claim 1, wherein the first and the second line generator comprise:
a first and a second source for forming a first and a second beam of radiation; and
first and second optical means for transforming the first and the second beam into the first and the second line.

3. The system recited in claim 2, wherein the first and the second optical means comprise:
a first and a second cylindrical lens downstream of the first and the second source; and
a first and a second mirror downstream of the first and the second cylindrical lens, respectively, the first and the second mirror oriented for forming a line from a beam emerging from the first and the second cylindrical lens.

4. The system recited in claim 3, further comprising a first and a second filter positioned between the first and the second source and the first and the second cylindrical lens.

5. The system recited in claim 1, further comprising a first and a second filter positioned between the first and the second source and the eye position.

6. The system recited in claim 1, wherein the adjusting means comprises means for moving a patient bed along a substantially vertical axis.

7. The system recited in claim 6, wherein the moving means comprises lifting means, motor means in activating contact with the lifting means, and control means in controlling relation to the motor means.

8. The system recited in claim 1, wherein the adjusting means comprises means for moving the laser surgical system relative to a patient bed.

9. The system recited in claim 1, wherein the adjusting means comprises means for achieving a positioning wherein the first and the second lines form a cross.

10. The system recited in claim 9, wherein the adjusting means comprises means for adjusting the eye position along an axis substantially perpendicular to the first and the second lines.

11. A method for positioning an eye for laser surgery comprising the steps of:
generating a first and a second line of radiation, the first line substantially orthogonal to the second line;
directing the first and the second lines to a position having a predetermined relation to a laser surgical system; and
adjusting the position of the first and the second lines relative to an eye to achieve a preferred position of the eye relative to the laser surgical system for laser surgery.

12. The method recited in claim 11, wherein the first and the second line generating step comprises:
forming a first and a second beam of radiation; and
transforming the first and the second beam into the first and the second line.

13. The method recited in claim 12, wherein the transforming step comprises:
inserting a first and a second cylindrical lens into the first and the second beam upstream of the eye position; and
inserting a first and a second mirror downstream of the first and the second cylindrical lens, respectively, the first and the second mirror oriented for forming a line from a beam emerging from the first and the second cylindrical lens.

14. The method recited in claim 13, further comprising the step of inserting a first and a second filter into the first and the second beam upstream of the first and the second cylindrical lens.

15. The method recited in claim 11, further comprising the step of inserting a first and a second filter in the radiation line upstream of the eye position.

16. The method recited in claim 11, wherein the adjusting step comprises moving a patient bed along a substantially vertical axis.

17. The method recited in claim 16, wherein the moving step comprises controlling a lifting means to move the patient bed.

18. The method recited in claim 11, wherein the adjusting step comprises moving the laser surgical system relative to a patient bed.

19. The method recited in claim 11, wherein the adjusting step comprises achieving a positioning wherein the first and the second lines form a cross.

20. The method recited in claim 19, wherein the adjusting step comprises adjusting the eye position along an axis substantially perpendicular to the first and the second lines.
